(19) United States
(12) Patent Application Publication
(10) Pub. No.: US 2004/0110848 A1
(43) Pub. Date: Jun. 10, 2004

(54) Method and kit for treating cancer

(76) Inventors: Dennis M. Peffley, Lee's Summit, MO (US); Patricia M. Hentosh, Lee's Summit, MO (US); Navesh K. Sharma, Lee's Summit, MO (US)

Correspondence Address: Law Offices of Loren K. Thompson, 1113-D Glendale, Topeka, KS, 66604, US

(21) Appl. No.: 10/315,352

(22) Filed: Dec. 10, 2002

(51) Int. Cl. A61K 31/045

(52) U.S. Cl. 514/739

(57) ABSTRACT

A method for treating cancer and associated kit are disclosed. The presently claimed method uses the strategy of inhibiting at least one biomechanical process associated with a carcinoma in a living organism to treat cancer by exposing the carcinoma to an effective amount of an isoprenoid, wherein said effective amount of isoprenoid inhibits the biomechanical process associated with the carcinoma.

### STATEMENT REGARDING FEDERALLY SPONSORED RESEARCH OR DEVELOPMENT

This invention was made with U.S. government support awarded by the following agencies: NIH Grant No.: R01-CA72527, “Dietary Isoprenoid Regulation of Growth Related Genes” funded through the National Cancer Institute; and R01-CA81756; Modulation of Mevalonate Synthesis by Dietary Isoprenoids” funded through the National Cancer Institute. The United States has certain rights to this invention.

### FIELD OF THE INVENTION

The present invention relates to a method and kit for use in inhibiting invasion, repressing extracellular matrix remodeling, and suppressing matrix metalloproteinase activity by tumor cells of living organisms.

### BACKGROUND

Prostate cancer is the most common non-skin malignancy in men, second only to lung cancer for cancer-related male deaths in the United States. There is a high prevalence of latent or occult prostate cancer in U.S. males over 50 years old. That is, post mortem studies have estimated that approximately 30 percent of males older than age 50 have histologic evidence of prostate cancer, in which it is even reported that some U.S. males, as early as 20 years of age, have detectable precursor lesions. Therefore, it is not surprising that the presence of microscopic adenocarcinoma foci in 30 to 50 year old U.S. males has been estimated to range from 25 to 32 percent. Yet, prostate cancer is relatively uncommon in male populations in many Asian countries.

Epidemiological studies have suggested that dietary intake of fruits and vegetables and other plant-related products may provide significant chemopreventive effects against hormone-related cancers. A number of micronutrients, in particular, β-carotene, ascorbic acid, α-tocopherol and folic acid, have been intensely studied to elucidate any corresponding chemopreventive effects that these micronutrients may convey when consumed. However, many of the results of these micronutrient studies have led to contradictory or inconclusive findings concerning their chemopreventive effectiveness.

A growing body of evidence indicates that anutrients, hereinafter defined as non-nutrient phytochemicals, such as anti-oxidants, dithiothriones, phenols, indoles, flavonoids, protease inhibitors and allium compounds, may also play key roles in either blocking or suppressing carcinogenic processes. Even though it is now generally considered that a wide variety of anutrients in plant-related diets is a primary contributor to chemoprevention, it is generally believed that a single anutrient compound is unlikely to be the sole cause of chemoprevention from these plant-related diets. Rather it appears that it is likely that multiple anutrient components impinge on multiple key cell growth signaling pathways simultaneously as the primary prevention mechanism of any cancer attributable to anutrients.

More recently, a subcategory of phytochemical anutrients, i.e., the secondary products of plant mevalonate metabolism, collectively defined herein as isoprenoids, have been recognized for their potential use in cancer prevention and treatment possibilities. Isoprenoid anutrient compounds are derived entirely or in part from the plant mevalonate biosynthetic pathway, which are further subcategorized into “pure” or “mixed” isoprenoids. Pure isoprenoids have varying structures consisting only of five-carbon isoprene units, e.g., monoterpenes, diterprenes, etc. Some important examples of pure isoprenoids include farnesol, limonene, perillyl alcohol, tocotrienols, ionone and taxol. Mixed isoprenoids include isoflavones, prenylated coumarins, flavones, flavanols, chalcones, quinones, and chromanols, each with only a part of the molecule derived via the mevalonate pathway. Some important examples of mixed isoprenoids include genistein, daidzein, lycopenes, and β-carotenes.

Soy-based products that contain mixed isoprenoids, in particular genistein and daidzein, may be linked to significant chemopreventive effects against hormone-related cancers. Genistein has demonstrated antiproliferative effects against a wide variety of tumor cells including breast cancer, leukemia and lymphoma, melanoma, lung cancer, and head and neck squamous carcinoma. Soy extracts rich in genistein have also been shown to suppress transplanted and chemically-induced prostate cancer cells in rodents. The molecular effects of genistein are multifaceted and include inhibition of tyrosine protein kinases, topoisomerases I and II, 5α-reductase, and protein histidine kinase as well as suppression of angiogenesis, growth factor stimulated responses, oncogene activity and prostaglandin synthesis. Genistein also induces a G2/M cell cycle arrest, which in turn suppresses cell growth. Other effects brought about by genistein include downregulation of cyclin B and upregulation of the growth-inhibitory protein p21WAF1. Treatment of the androgen-responsive prostate tumor cell line LNCaP, with genistein concentrations above 20 μM has been shown to induce apoptosis, in which this response seems most likely associated with increased p21WAF1 expression. Overall, reports have shown that genistein can inhibit growth of human prostate cancer cells in culture, but generally at supraphysiological concentrations above 50 μmol. Unfortunately, the effects of isoprenoids with or without genistein at physiological concentrations on prostate cancer metastasis have not been explored. Because metastatic prostate cancer is considered incurable, pre-metastatic treatment, such as nutritional suppression of both tumor cell growth and invasive potential may provide a more effective chemopreventive strategy.

In plants, both pure and mixed isoprenoids function in growth regulation, in host defense systems against insects and act as chemoattractants. In mammalian cells, many of these same isoprenoid compounds directly or indirectly regulate mammalian mevalonate biosynthesis through a mechanism that modulates 3-hydroxy-3-methylglutaryl coenzyme A (HMG-CoA) reductase, which is the major rate limiting enzyme of mevalonate synthesis. As depicted in FIG. 1, isoprenoid products derived from mevalonate via the cholesterol biosynthetic pathway in mammalian cells, e.g., ubiquinone, dolichol, isopentenyl tRNA, farnesyl and geranylgeranyl for protein isoprenylation, and cholesterol, are all essential for normal cell growth, for maintenance of cholesterol homeostasis, and for posttranslational modification and biological activity of more than 50 known proteins including p21ras, nuclear lamins A and B and growth factor receptors. This finely tuned metabolic feedback mechanism involves transcriptional as well as post-transcriptional inputs which maintains the pool of mevalonate pathway intermediates mentioned above that are important for normal cell survival and homeostasis.

As mentioned above and illustrated in FIG. 1, HMG-CoA reductase is the major rate-limiting enzyme of the mevalonate/cholesterol pathway in mammalian cells, in which the HMG-CoA reductase is finely regulated in normal cells at the transcriptional, translational, and degradation levels to maintain the pool of mevalonate intermediates. For example, cellular cholesterol controls HMG-CoA reductase mRNA transcription via binding of the sterol regulatory element binding protein (SREBP) to the sterol response element in the HMG-CoA reductase gene promoter. In contrast, tumor cell growth and survival are highly dependent on attenuation of sterol (cholesterol)-mediated HMG-CoA reductase gene regulation, a phenomenon referred to as sterol-independent regulation of HMG-CoA reductase. As a consequence, HMG-CoA reductase levels are elevated in many tumor cells and cell lines, and translational efficiency of these transcripts is increased to ensure adequate isoprenoid production in the cholesterol-rich environment of the tumor.

Regulation of HMG-CoA reductase at the transcriptional level is affected by a number of sterols, e.g., cholesterol and/or oxygenated sterol metabolites. These sterols act through a sterol response element (SRE)—GTGCGGTG—in the 5′-promoter region of the HMG-CoA reductase gene to control HMG-CoA reductase mRNA transcription. Sterol-dependent regulation of transcription is mediated through binding of the SREBP to the SRE. Two SREBP isoforms are produced from the SREBP-1 gene, SREBP-1a and SREBP-1c. A third major isoform, SREBP-2, is derived from the SREBP-2 gene. SREBP-1a and SREBP-2 are primarily associated with regulating sterol or cholesterol-responsive genes, while SREBP-1c primarily regulates genes involved in fatty acid synthesis. Through this process, cellular cholesterol coordinately regulates multiple genes involved in cholesterol biosynthesis and uptake. SREBP itself is also exquisitely regulated. SREBP precursors are attached to the endoplasmic reticulum (ER) and nuclear envelope. When cells are depleted of cholesterol, the membrane-bound 125-kDa SREBP precursor is cleaved to generate a soluble 68-kDa N-terminal fragment that translocates to the nucleus and activates transcription of genes involved in both cholesterol and fatty acid metabolism through binding to the SRE. The SREBP cleavage-activating protein (SCAP) enhances SREBP exit from the ER membrane. In contrast, high sterol levels normally block exit of a SCAP/SREBP complex from the ER thus preventing the cleavage step and attenuating SREBP processing. Because SREBP regulates transcription of HMG-CoA reductase, as well as HMG-CoA synthase, farnesyl diphosphate synthase, squalene synthase, low density lipoprotein receptor (LDL-R) levels, plus two enzymes involved in fatty acid synthesis—fatty acid synthase and acetyl coenzyme A carboxylase—cholesterol-mediated regulation of SREBP proteolysis allows for coordinate control of genes involved in cholesterolgenesis and fatty acid metabolism. Additional co-regulatory transcription factors are required for high activation levels, and there are also negative regulators of SREB P-mediated transcription.

Translational regulation of HMG-CoA reductase occurs at the level of initiation, which is usually the rate-limiting step in cellular translation processes; thus the rate of reductase mRNA translation is determined by how efficiently the initiation complex forms at the 5′ cap of mRNA. Mevalonate-derived isoprenoids regulate reductase gene initiation by reducing the translational efficiency of reductase mRNA. Specifically, isoprenoids reduce the amount of reductase mRNA associated with heavier polyribosomes. Sterols do not seem to act as control factors at the translational stage. We previously established that the 5′-untranslated leader (UTL) region of reductase mRNA is required for isoprenoid-mediated translational regulation, an effect mediated through the cap binding protein, initiation factor 4E (eIF4E).

Regulation of HMG-CoA reductase at the post-translational level occurs through protein degradation that is mediated by both sterols and mevalonate-derived isoprenoids. This process requires the intact ER membrane-spanning region of reductase, which consists of multiple hydrophobic domains. If the membrane-associated domains are deleted, neither sterols nor mevalonate affect reductase turnover. Farnesol, a mevalonate derivative, was identified as a regulatory factor for the accelerated degradation of reductase protein. Thus farnesol regulates reductase at two levels. It has recently been shown that both mammalian and yeast reductase undergo regulated degradation through the ubiquitin-proteosome pathway.

HMG-CoA reductase also regulates tumor cell viability. The mevalonate pathway is not only important for regulating cholesterol production but has an equally important role in providing many small signaling molecules that trigger cellular events including cell proliferation and death. Isoprenoids profoundly alter the ability of tumor cells to synthesize mevalonate and these mevalonate-derived signaling molecules. Consequently, isoprenoids have the ability to either directly or indirectly modify signal transduction pathways that control cell growth and death.

Aberrantly elevated and feedback-resistant mevalonate activities in tumor tissue were recognized in the 1950's by researchers who proposed that a fundamental lesion of malignant cells might be a feedback control defect that allowed uninhibited synthesis of a key growth intermediate. Shortly thereafter, the dysregulation of cholesterol synthesis was reported in tumors and tumor-bearing animals. Others have observed the unique sensitivity of tumor cells and aberrant mevalonate pathway activities to dietary isoprenoids. Yet others found that isoprenoids extracted from barley—the tocotrienols—suppressed cholesterol synthesis. Previous studies in our laboratories have focused on the molecular and cytotoxic effects of various “pure isoprenoids”. Specifically we found that tumor-associated HMG-CoA reductase levels remain highly sensitive to isoprenoid—mediated translational suppression. In addition we and others have established that tumor cells undergo an apoptotic-like cell death when treated with isoprenoids, an effect mediated in part through inhibition of nuclear lamin farnesylation and processing. Isoprenoid effects are analogous to those of HMG-CoA reductase inhibitors such as lovastatin and simvastatin, that have antiproliferative effects on tumor cells by arresting cell growth in the G1 phase of the cell cycle. This results primarily from decreased levels of mevalonate-derived isoprenoids required for farnesylation and geranylation of proteins involved in mitogen-activated cell growth. Further, inhibition of post-translational farnesylation of p21Ras oncogene and nuclear lamins has been shown to induce apoptosis and suppress cell growth.

Mitogenic stimulation influences mevalonate and cholesterol biosynthesis. The putative link(s) between caveolae-associated signal transduction proteins and HMG-CoA reductase regulation is shown in FIG. 2. Cell transformation is frequently associated with constitutive activation of components in these signal transduction pathways that control cell proliferation and differentiation. Increased activation of these representative signal transduction pathways regulates gene expression that is required to initiate and maintain rapid cell proliferation characteristic of tumor cells. These pathways are initiated by receptor tyrosine kinases, cytokine receptors and G proteins, all of which mediate activation of intracellular protein serine/threonine kinases termed mitogen-activated protein kinases (MAPKs), also known as extracellular signal-related kinases (ERKs). FIG. 2 depicts two common pathways leading from cell surface receptors to MAPKS. Activation of tyrosine kinases results in the recruitment of SH2 (Src homology) domain-containing proteins that include the p85 regulatory subunit of phosphoinositide 3-OH (P13) kinase and the guanine-nucleotide exchange factor Grb2/SOS. The pathway depicted on the right in FIG. 2 involves the small GTP-binding protein p21 Ras, which binds to c-Raf protein kinase and leads to its activation. Raf phosphorylates and activates MAP Kinase Kinase (MAPKK), or MEK, which then phosphorylates and activates MAPKs. In addition, MAPKs activation also occurs through PI3-K in the pathway depicted on the left in FIG. 2. Translocation of MAPKs (or ERKs) to the nucleus then regulates numerous transcription factors such as c-fos c-myc, and SREBP.

The effect of mitogenic stimulation on regulation of cellular cholesterol levels has been studied primarily in relation to the LDL-R gene. LDL-R gene expression is increased by a number of non-sterol signaling molecules such as growth factors, cytokines, and calcium ionophores in several cell lines. It has been reported that mitogen-stimulation of a human leukemic T cell line in the absence of sterols increases nuclear SREBP accumulation. Recent studies have demonstrated that the extracellularly responsive kinases [p44/p42 MAPK or (ERK)-1/2] regulate sterol-mediated gene transcription. Part of this regulation may be attributed to a direct link between SREBP-1a and SREBP-2 and p44/p42 MAPK. Others have demonstrated that upstream activators of MAP kinases, MEKK1 or MEK1, stimulated LDL-R promoter activity several fold in an SRE-1 related manner. Experiments also indicate that ERK 1/2 phosphorylates SREBP-1a and SREBP-2 in vitro suggesting that these transcription factors may be substrates for ERK1/2 within a cell. In view of these findings, sterol-independent regulation of both HMG-CoA reductase and LDL-R genes may be due in part to ERK 1/2 activation of SREBP.

Elevated levels of receptor tyrosine kinase have been implicated in a wide range of malignancies including prostate cancer. Compelling evidence for the link between tyrosine kinases and sterol-independent regulation of HMG-CoA reductase was recently described. Tyrosine kinase inhibition by herbimycin A in human breast adenocarcinoma SKBR-3 cells significantly suppressed HMG-CoA reductase mRNA levels and synthesis. It has not been established if the tyrosine kinase effect on HMG-CoA reductase mRNA is strictly at the transcriptional level or also involves effects on mRNA stability. Additionally, it was found that epidermal growth factor (EGF) mediated stimulation of HMG-CoA reductase occurred only in breast cancer cells overexpressing ErbB-2, an EGF receptor with ligand-activated tyrosine kinase activity. At the present time, it is not known if this effect is mediated via enhanced SREBP-2 expression as well as accelerated proteolytic processing of the ER-associated form. Moreover, it has been shown that genistein treatment of DU145 prostate cancer cells inhibits downstream signaling targets through a EGFR-She-Grb2/SOS-ras-raf-ERK1/2 (MAPK) signaling cascade (FIG. 2) that results in inhibition of ERK 1/2 (MAPK)-mediated mitogenic signaling. That study, however, did not investigate HMG-CoA reductase or SREBP levels. Others have reported that in LNCaP prostate tumor cells, androgens increased steady state mRNA levels of fatty acid synthase (FAS) via a pathway involving SREBP-1. Moreover, EGF stimulated FAS mRNA, protein and activity levels as well as the amount of mature nuclear-associated SREBP-1c, the SREBP isoform primarily involved in fatty acid synthase regulation. EGF-mediated increases in FAS mRNA were determined to be dependent on the 178 bp FAS promoter region containing the complex SREBP-binding site.

SREBP, androgenic stimulation and prostate cancer are known to be related. First, others have shown that human prostate cancer cells, PC-3 and DU145, lack feedback regulation of low density lipoprotein and its regulator, SREBP-2, compared to normal human prostate epithelial cells. The failure to suppress SREBP-2 suggests that HMG-CoA reductase and other sterol-responsive genes would also display sterol-independent regulation in prostate tumor cells. Second, still others reported that androgens increased both SREBP-2 and SREBP-1 levels and, as a result, steady-state mRNA levels of seven different enzymes belonging to two major lipogenic pathways, i.e., fatty acid synthesis and cholesterol synthesis, were elevated. Included among these was HMG-CoA reductase. Promoter based studies indicated that this effect was mediated at the transcriptional level through the SRE. Third, yet others demonstrated that androgens mediate their effect on target genes in part through the HER2/Neu-MAP kinase-Androgen Receptor (AR)/Androgen Receptor Coactivator (ARA) pathway. HER2/Neu (erbB2) is a member of the class I receptor tyrosine kinase family or the epidermal growth factor receptor (EGFR). The EGFR family represents the most frequently implicated receptor in human cancer. This association between androgen-mediated increases in SREBP and class I receptor tyrosine kinases provides convincing theoretical support that prevention and management of prostate cancer is possible by targeting mevalonate synthesis. Specifically, by inhibiting tyrosine kinase activity with genistein, we expect to suppress mevalonate synthesis effectively by reducing SREBP expression in both androgen-dependent and androgen-independent cells, and effectively suppress their growth as well as induce apoptosis.

In addition to regulating transcription factor activity in the nucleus, mitogenic activation of these transduction pathways affects phosphorylation of key initiation factors involved in regulating protein synthesis. The rate limiting translation initiation factor is eukaryotic initiation factor eIF4E, also known as the cap-binding protein (FIG. 3). The eIF4E is responsible for binding to the 5′-terminal 7-methyl-GTP (m⁷GTP) cap found on all eukaryotic mRNAs. The eIF4E is part of the cap-binding complex eIF4F, along with eIF4A and eIF4G. Levels of eIF4E available for eIF4F formation are tightly regulated in normal cells. In quiescent cells, most eIF4E is associated with its inhibitor, PHAS or 4E-BP1. In response to mitogenic stimulation, 4E-BP1 becomes heavily phosphorylated via the P13 kinase, mTOR and releases eIF4E. This results in a small increase for basal protein synthesis but has a markedly greater stimulation of translation from specific mRNAs coding for growth promoting factors including ornithine decarboxylase, c-myc, cyclin D1, vascular endothelial growth factor and ribonucleotide reductase. In addition, the present invention demonstrates that translation of HMG-CoA reductase mRNA is similarly increased by eIF4E (see FIG. 5). All these mRNAs have as a common feature 5′-untranslated leader (5′-UTL) sequences that are GC rich and contain extensive secondary structure. Mitogenic activation can phosphorylate eIF4E, a posttranslational modification that is associated with increased binding of eIF4E to capped mRNA and to eIF4F in vitro. The eIF4E phosphorylation is mediated in vivo through the protein kinase mitogen- and stress-activated kinase, Mnkl. Specific translational regulation of mRNAs also occurs through mTOR-mediated activation of p70s6 kinase (p70s6k) (see FIG. 3). The substrate for p70s6k is s6 ribosomal protein; s6 phosphorylation results in enhanced binding affinity of ribosomes to AUG sites. This results in not only a global increase in protein synthesis but also results in selective increases in translation of mRNAs having a polypyrimidine tract of 5-14 bases followed by a cytosine in the transcriptional start site, close to the cap structure. The concomitant involvement of this signaling pathway in both HMG-CoA reductase regulation and isoprenoid sensitivity further supports the present invention scheme of targeting mevalonate production in prostate tumor cells. That is, by targeting two effectors, i.e., eIF4E and SREBP, in these signal transduction pathways, the utility of the present invention towards treating tumor cells extends well beyond merely affecting HMG-CoA reductase. Other growth promoting genes that are regulated by either of these two proteins, e.g., ornithine decarboxylase, c-myc, ribonucleotide reductase, fatty acid synthase, LDL-R, HMG-CoA synthase and others are also negatively impacted and contribute to growth suppression and/or killing of transformed prostate cells.

Tumor cell metastasis is one step in the metastatic cascade and is mediated in part by proteolytic enzymes called matrix metalloproteinases (MMPs) and lysosomal proteases (cathepsins). MMPs are a family of enzymes which are intimately involved in the degradation and remodeling of connective tissues. MMPs are found in a number of cell types that are associated with connective tissue, such as fibroblasts, monocytes, macrophages, endothelial cells and metastatic tumor cells. They also share a number of properties, including zinc and calcium dependence, secretion as zymogens, and 40-50% amino acid sequence homology. MMPs are known to degrade protein components of the extracellular matrix, i.e. the protein components found in the linings of joints, interstitial connective tissue, basement membranes, cartilage and the like. These proteins include collagen, proteoglycan, fibronectin and laminin. Since collagen is the major structural protein of mammalian tissue, comprising one-third of the total protein in mammalian organisms, it is an essential component of many matrix tissues, including cartilage, bone, tendons and skin. Interstitial collagenases catalyze the initial (rate-limiting) cleavage of native collagen types I, II, III and X. MMP enzymes are known to cleave collagen into two fragments which subsequently spontaneously denature further at physiological temperature. The net effect of MMP enzyme initiated collagenase cleavage is the loss of structural integrity in the matrix tissue (collagen collapse), an essentially irreversible process. In normal tissues, the activity of MMPs is tightly regulated. As a result, the breakdown of connective tissue mediated by these enzymes is generally in a dynamic steady state balance with the synthesis of new matrix tissue. However, in a number of pathological disease conditions, deregulation of MMP activity leads to the uncontrolled breakdown of extracellular matrix. These disease conditions include arthritis (e.g., rheumatoid arthritis and osteoarthritis), periodontal disease, aberrant angiogenesis, tumor metastasis and invasion, tissue ulceration (e.g., corneal ulceration, gastric ulceration or epidermal ulceration), bone disease, HIV-infection and complications from diabetes.

MMPs represent a family of secreted or membrane-associated proteins that degrade extracellular matrix and basement membrane elements. Various human prostate tumor cell lines are known to express MMP-2 (gelatinase A), MMP-9 (gelatinase B), MMP-7 (matrilysin) and hyaluronidase. Others have reported increased incidence of both MMP-2 and MMP-9 in urine from patients with a variety of cancers. MMPs are highly regulated and controlled at the transcriptional (mRNA) level, at the translational level (as shown in murine prostate carcinoma cells), at the level of secretion an activation. There is also evidence that MMPs are shed as MMP-containing vesicles. In head and neck cancer cell lines, genistein was shown to down-regulate MMP-2 and MMP-9 secretion and inhibited tumor cell invasion. At least three signaling pathways, i.e., PKC, p38 kinase, and Mek-1, are thought to be involved in heregulin-β1 activated MMP-9 in human breast cancer cell lines. PKC and Ras/Erk (Mek1/2) signaling pathways are also reported to be involved in MMP-9 secretion in MCF-7 cells. Prior to the present invention, the effects of isoprenoids with or without genistein on prostate cancer have not been explored. The present invention provides compelling support that these nutritional supplements have a significant effect on MMP activity. Because metastatic prostate cancer is considered incurable, nutritional suppression of both tumor cell growth and invasive potential may provide a more effective chemoprevention strategy.

An intriguing link between the mevalonate pathway and metastasis involves the Rab family of small GTP-binding proteins, which are important regulators of vesicle trafficking and exocytosis in eukaryotic cells. Most Rabs have a geranylgeranylation moiety, derived from mevalonate, at the carboxyl terminus that functions in anchoring or tethering vesicles to an acceptor membrane, and in both protein:protein and protein:lipid interactions. A prenylation-deficient Rab4 exhibited decreased Glut4 translocation and reduced Akt activation in response to insulin. Moreover, Rab5A overexpression has been correlated with metastatic potential in human lung cancer cell lines. Rab5 is thought to sequester ligands at plasma membranes through a tethering mechanism. Equally important are Rab3 members that are expressed in many epithelial cells and appear to be involved in protein secretion. In response to the epidermal growth factor, heregulin, Rab3A mRNA and protein were found to be up-regulated in human breast epithelial cells, as well as to increase membrane-bound Rab3A levels. Secretion of cellular proteins likewise was enhanced. Conversely, bisphosphonate analogs, which act via the mevalonate pathway, selectively prevented cellular Rab geranylgeranylation but not that of Ras or Rap, and disrupted Rab-dependent intracellular membrane trafficking. The present invention reveals that Rab3A is present at high levels in metastatic prostate cancer cell lines, but is undetectable in normal prostate cells and in Caco-2 colon carcinoma cells that have low metastatic ability. This raises the novel possibility that Rab-mediated tethering of vesicles containing or transporting MMPs to plasma membranes are targets of isoprenoids through reduced prenylation.

Therefore, it is not surprising that a large number of studies have established the anti-tumorigenic properties of pure isoprenoids. It has been clearly shown that isoprenoids, e.g., limonene, perillyl alcohol, γ-tocotrienol, β-ionone, and farnesol, initiate apoptosis and concomitantly arrest tumor cells in the G1 phase of the cell cycle. Tocotrienols have been shown to be especially effective at inhibiting growth of both murine and human breast cancer cells in culture. Pure and mixed isoprenoids have been shown to suppress growth of a vast number of whole animal tumor models including implanted leukemic cells, melanomas, pancreatic tumors and hepatomas.

Since many forms of metastatic cancer are considered incurable, in particular prostate cancer, then pre-metastatic treatment methods and associated compounds aimed at inhibiting various biomechanical processes associated with carcinomas, such as overproduction of HMG-CoA reductase mRNA, enhanced translation of HMG-CoA reductase from its mRNA template, the elevated 4E-BP1 phosphorylation, the over-expression of eIF4E, the increased levels and activity of matrix metalloproteinases (MMPs), the elevated Rab3A levels, extracellular matrix remodeling, and secondary tissue invasion by cancer cells offer novel therapeutic tactics in controlling these cancers. Therefore, there is a need to identify new and useful methods to treat various cancers to inhibit associated biomechanical processes common in many carcinomas.

### SUMMARY OF THE INVENTION

The present methods of treating cancer and the associated kits for using these methods, according to the principles of the present invention, overcome the shortcomings of the prior art by providing methods of treating cancer by inhibiting at least one biomechanical process associated with a carcinoma in a living organism, said method comprising the step of exposing the carcinoma to an effective amount of an isoprenoid, wherein said effective amount of isoprenoid inhibits the biomechanical process associated with the carcinoma. Accordingly, these biomechanical processes associated with carcinoma which are inhibited by the present invention comprise, and are not limited to, the overproduction of HMG-CoA mRNA, the enhanced translation of HMG-CoA reductase from its mRNA template, the elevated 4E-BP1 phosphorylation, the overexpression of eIF4E, the increased levels and activity of matrix metalloproteinases (MMPs), the elevated Rab3A levels, extracellular matrix remodeling, and secondary tissue invasion.

By the terms “treatment” or “treating”, as used herein, we mean inhibiting the growth of the carcinoma, controlling the growth of the carcinoma, reducing the mass of the carcinoma, eliminating the carcinoma, preventing the carcinoma from being established in the living organism, or repressing the carcinoma from advancing to a secondary carcinoma.

By the term “exposing”, as used herein, we mean contacting directly the isoprenoid onto the carcinoma in the living organism, administering the isoprenoid intravenously in the living organism, injecting the isoprenoid intraperitoneally in the living organism, applying the isoprenoid subcutaneously in the living organism, inserting the isoprenoid intramuscularly in the living organism, employing the isoprenoid intrathecally in the living organism, swallowing the isoprenoid orally by the living organism, introducing the isoprenoid rectally into the living organism, rubbing the isoprenoid topically onto the living organism, and inhaling the isoprenoid by the living organism.

By the term “mammal”, as used herein, we mean all mammalian species, such as, monkeys, horses, pigs, goats, sheep, dogs, and cats. More preferable, the methods and associated kits of the present invention are intended to be used for the treatment of primates. Most preferably, the methods and associated kits of the present invention are intended to be used for the treatment of humans.

By the term “cell toxicity”, we mean an adverse chemical effect on normal (noncancerous) cells which are sufficient to cause death of normal cells. By excessive toxicity is meant adverse effects on normal (noncancerous) cells which are sufficient to cause the death of the living organism.

By the term “cancer”, as used herein, we mean any carcinoma, pre-carcinoma condition, and metastatic carcinoma condition. More preferable, the methods and associated kits of the present invention are intended to be used for the treatment of cancers selected from the group consisting of cancers of the central nervous system, gastrointestinal tract, epidermal system, head and neck system, genitourinary tract, lymphatic system, cardiovascular system, hepatic system and respiratory system.

By the term “isoprenoid”, as used herein, we mean a member of a “pure” or a “mixed” isoprenoid. Pure isoprenoids have varying structures consisting only of five-carbon isoprene units, e.g., monoterpenes, diterprenes, etc. Some important examples of pure isoprenoids of the present invention include farnesol, limonene, perillyl alcohol, tocotrienols, ionone and taxol. Some important ionone pure isoprenoids of the present invention are selected from the group consisting of 4-(2,6,6-trimethyl-1-cyclohexen-1-yl)-3 -buten-2-one; 4-(2,6,6-trimethyl-2-cyclohexen-1-yl)-3-buten-2-one; 6,10-dimethyl-9,10,-epoxy-undec-3,5-ene-2-one; 9,10-diacetoxy-6,10-dimethyl-undec-3,5-ene-2-one; and 6,10-dimethyl-9,10-diol-undec-3,5-ene-2-one. Some important tocotrienol pure isoprenoids of the present invention are selected from the group consisting of 2,5,7,8-trimethyl-2-(4,8,12-trimethyltrideca-3,7,11-trienyl)-chroman-6-ol; 2,5,8-trimethyl-2-(4,8,12-trimethyltrideca-3,7,11-trienyl)-chroman-6-ol; 2,7,8-trimethyl-2-(4,8,12-trimethyltrideca-3,7,11-trienyl)-chroman-6-ol; and 2,8-dimethyl-2-(4,8,12-trimethyltrideca-3,7,11-trienyl)-chroman-6-ol. Mixed isoprenoids include, isoflavones, prenylated coumarins, flavones, flavanols, chalcones, quinones, and chromanols, each with only a part of the molecule derived via the mevalonate pathway. Some important examples of mixed isoprenoids intended for use in the present invention are selected from the group consisting of genistein, daidzein, lycopenes, and β-carotenes.

By the term “additive”, as used herein, we mean a percentage reduction in cell number corresponding to the additive sum of individual effects or an additive sum of individual effects to a host survivability.

By the term “synergistic”, as used herein, we mean a percentage reduction in cell number of at least an additional 5% over the additive sum of individual effects or an increase in host survivability of 5% of the additive sum of individual effects.

By the term “antagonistic”, as used herein, we mean a percentage increase in cell number of at least an additional 5% over the additive sum of the individual effects or a decrease of host survivability of 5% of the additive sum of individual effects.

By the term “effective amount”, as used herein, we mean a dosage capable of inhibiting any one of the biomechanical processes associated with the carcinoma, referred to in this present invention, without subjecting the living organism to adverse chemical effects on normal (noncancerous) cells such as those dosages which are sufficient to cause death of normal cells. Wherein the dosage for treating a living organism against cancer in accordance with the present invention will be in the range of about 1 nanogram to about fifty grams daily for the isoprenoid. Exact dosages will depend on the extent to which the compounds are metabolized as well as their bioavailability to the target tissue. Appropriate doses in individual cases can be determined by persons of ordinary skill in the art.

By the term “delivery device”, as used herein, we mean any known commercially available vehicle capable of delivering the effective amount of the isoprenoid. These delivery devices may be selected from the group consisting of a tablet, a capsule, a solution, a suspension, an emulsion, a foodstuff, a pharmaceutical preparation, a nutritional supplement, and a dietary additive.

In view of the foregoing disadvantages inherent in the known types of methods for treating living organisms against cancer now present in the prior art, the present invention provides an improved methodology strategy of inhibiting at least one biomechanical process associated with a carcinoma in a living organism, which will be described subsequently in great detail, and a new and improved kit associated with the present method invention of exposing the carcinoma to an effective amount of an isoprenoid, wherein said effective amount of isoprenoid inhibits the biomechanical process associated with the carcinoma which are not anticipated, rendered obvious, suggested, or even implied by the prior art, either alone or in any combination thereof.

To attain this, the present method invention of inhibiting at least one biomechanical process associated with a carcinoma in a living organism essentially comprises the step of exposing the carcinoma to an effective amount of an isoprenoid, wherein said effective amount of isoprenoid inhibits the biomechanical process associated with the carcinoma.

There has thus been outlined, rather broadly, the more important features of the invention in order that the detailed description thereof that follows may be better understood, and in order that the present contribution of the art may be better appreciated.

Numerous aspects, features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an aspect of the present invention to provide a new and improved method of treating cancer to suppress activity of at least one metastatic biomechanical process exhibited from a tumor carcinoma in a living organism by exposing the carcinoma to an anti-neoplastic effective amount of an isoprenoid.

It is another aspect of the present invention to provide a kit for using a method of treating cancer to suppress activity of at least one metastatic biomechanical process exhibited from a tumor carcinoma in a living organism by exposing the carcinoma to an anti-neoplastic effective amount of an isoprenoid.

An even further aspect of the present invention is to provide a new and improved kit for using a method of treating cancer to suppress activity of at least one metastatic biomechanical process exhibited from a tumor carcinoma in a living organism that has a low cost of manufacture with regard to both materials and labor, and which accordingly is then susceptible to low prices of sale to the consuming public, thereby making such kit economically available to the buying public.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be madeto the accompanying drawings and description matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a proposed model depicting the mevalonate and cholesterol biosynthetic pathway in mammalian living organisms;

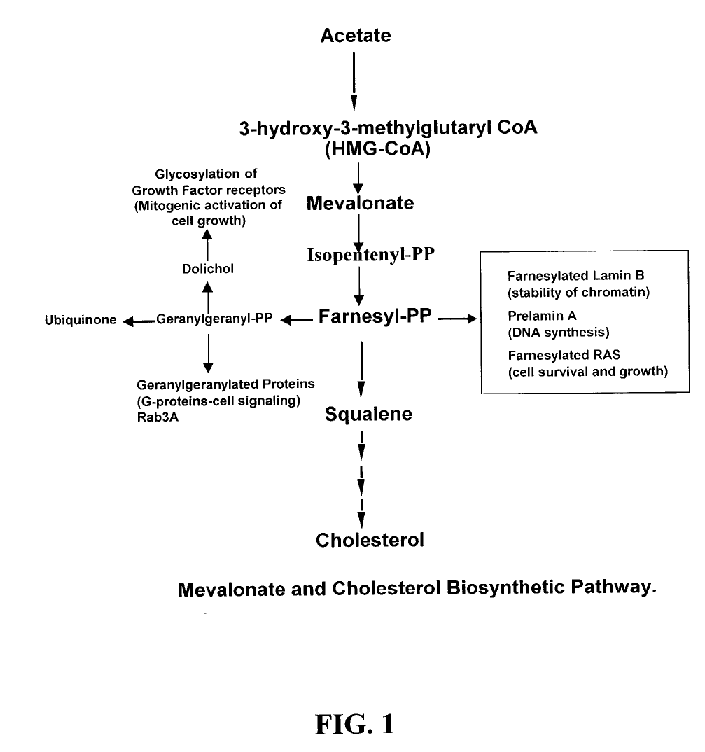

FIG. 2 is a schematic representation of a putative relationship of signal transduction pathways to sterol-mediated gene regulation;

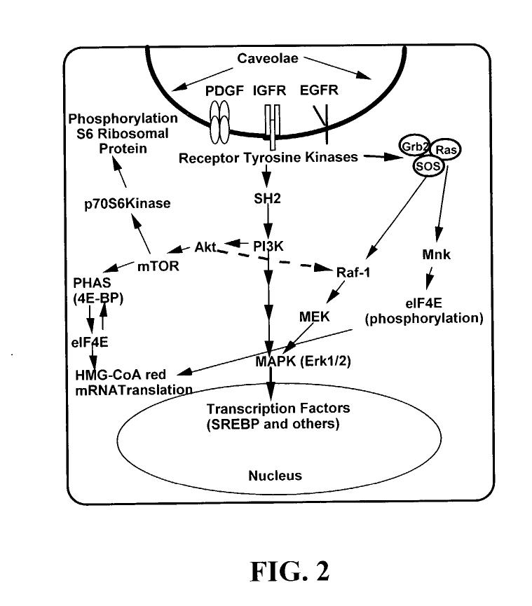

FIG. 3 is a schematic relationship of the pathway for mitogen activated protein synthesis to eIF4E-mediated regulation of HMG-CoA reductase synthesis;

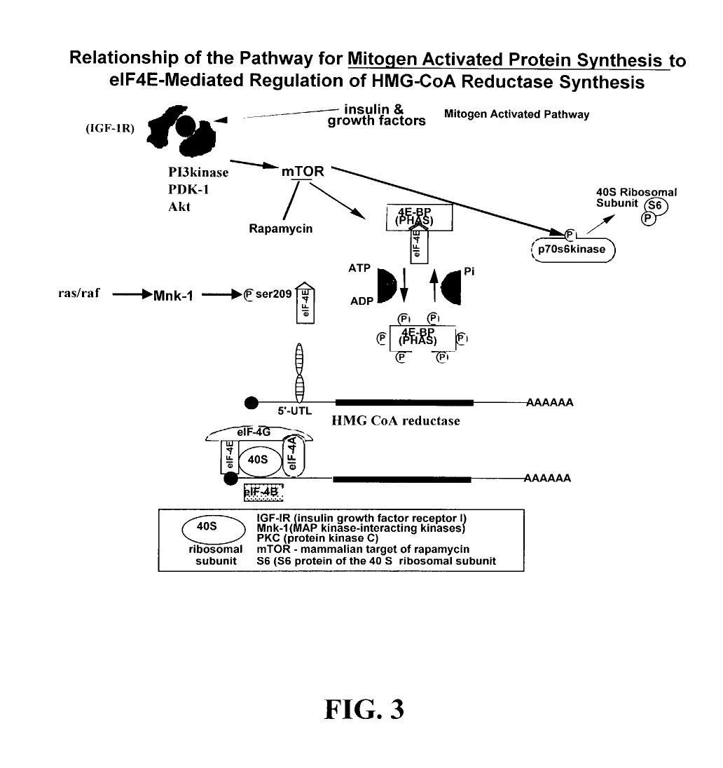

FIG. 4 is a bar diagram of adjusted luciferase counts per minute associated with CCD18 and Caco-2 cell lines;

FIG. 5 is an autoradiograph of ³⁵S-labeled HMG-CoA reductase immunoprecipitate;

FIG. 6 is a Western blot analysis of eIF4E, 4E-BP1 (Ser65) and Rab3A from various cells before and after various treatment conditions;

FIG. 7 is a Western blot analysis of eIF4E in PC-3, DU145 and PrEC cells after various treatments;

FIG. 8 is a gelatin zymogram showing the effects of perillyl alcohol, genistein, or γ-tocotrienol on MMP activity; and

FIG. 9 is a set of photographs of in vitro collagen gel contraction assay illustrating effects of isoprenoids on a PC3 prostate cancer cell line.

The same reference numerals refer to the same parts throughout the various figures.

### DESCRIPTION OF THE INVENTION

In one preferred embodiment, the present invention comprises methods of treating cancer by inhibiting at least one biomechanical process associated with a carcinoma in a living organism, in which the method comprises the step of exposing the carcinoma to an effective amount of an isoprenoid, wherein the effective amount of isoprenoid inhibits the biomechanical process associated with the carcinoma. Accordingly, these biomechanical processes associated with carcinoma which are inhibited by the present invention comprise, and are not limited to, the overproduction of HMG-CoA mRNA, the enhanced translation of HMG-CoA reductase from its mRNA template, the elevated 4E-BP1 phosphorylation, the overexpression of eIF4E, the increased levels and activity of matrix metalloproteinases (MMPs), the elevated Rab3A levels, extracellular matrix remodeling, and secondary tissue invasion.

### EXAMPLE 1

All cell cultures were maintained at 37° C. in a 5% CO₂-humidified environment. The three human prostate cancer cell lines, LNCaP, PC-3 and DU145 (American Type Culture Collection) were maintained as monolayer cultures in RPMI 1640 supplemented with 10% FBS, glutamine and antibiotics.

Prostate tumor cells were found to have significantly higher levels of HMG-CoA reductase. By means of competitive reverse transcriptase-polymerase chain reaction (RT-PCR), we have measured elevated levels of HMG-CoA reductase mRNA in two human prostate cancer cell lines, PC-3 and DU145. Briefly, total cellular RNA from 5×10⁶ cells was prepared using Trizol Reagent according to manufacturer's protocols followed by treatment with RNase-free DNase. RNA was phenol:chloroform extracted, ethanol-precipitated, resuspended in DEPC-treated water, and its concentration determined by UV-spectroscopy. Cellular HMG-CoA reductase mRNA was measured by using a GeneAmp® RNA PCR kit, 1 μg total RNA and pAW109 mimic RNA (Perkin Elmer) as an internal standard. pAW109 is a synthetic RNA that has complementary primer sequences for human HMG-CoA reductase. Cellular RNA and varying amounts of mimic pAW109 RNA (from 2×10⁶ to 1×10⁴ molecules/reaction) were co-reverse transcribed into cDNA with random hexamer primers and Murine Leukemia Virus reverse transcriptase (MuLV-RT). To each cDNA mixture was added Taq DNA polymerase in 1× PCR buffer and a HMG-CoA reductase gene-specific primer set. PCR products were analyzed by agarose gel electrophoresis and visualized by ethidium-bromide staining. Co-amplification of mimic and cellular cDNA yielded two products of different sizes. The relative amounts of each cDNA species were quantified by scanning DNA bands on a Kodak Image Station. Data from several gels were quantitated by computer imaging, and band optical density readings were obtained and plotted. These studies revealed that PC-3 had approximately 2×10⁶ copies of HMG-CoA reductase mRNA/μg of cellular RNA; DU145 had approximately 5.1×10⁶ copies of HMG-CoA reductase mRNA/μg of cellular RNA. In contrast, normal prostate epithelial cells (PrEC) were found to have significantly lower levels at approximately 1×10⁶ copies of HMG-CoA reductase mRNA/μg of cellular RNA, in the presence of 10% FBS.

### EXAMPLE 2

We investigated whether enhanced HMG-CoA reductase mRNA levels resulted from increased or novel transcription factor binding to the promoter region. It was found that tumor cells have elevated HMG-CoA reductase promoter activity.

HMG-CoA reductase promoter activity in normal and tumor cells was assessed by transiently transfecting a luciferase reporter gene construct, pRedLuc, into CCD 18 and colon adenocarcinoma cells (Caco-2). We had previously demonstrated elevated HMG-CoA reductase mRNA levels in Caco-2 compared to normal CCD18 cells, in a similar manner as seen in human prostate tumor cells. The plasmid vector, pRedLuc, contains Chinese hamster HMG-CoA reductase promoter sequences from −277 to +20 fused to a firefly luciferase coding sequence. This promoter includes a SRE and promoter factor 2 region that are homologous to SREBP and NF-1 transcription factor binding regions in the human HMG-CoA reductase promoter, respectively. Firefly luciferase activity provides a convenient and sensitive mode to assess promoter activity in different cell types. One million cells were transiently transfected with pRedLuc DNA using DOTAP liposomal reagent. As an internal control to measure transfection efficiency, pRL-SV40 vector, which contains cDNA encoding Renilla luciferase under a non-sterol regulated SV40 early promoter and enhancer, was co-transfected with pRedLuc. Transfected cells were incubated for 24 hr before harvesting. The activity of control Renilla luciferase and firefly luciferase, which represents HMG-CoA reductase promoter activity, was measured by scintillation counting using a Dual-Luciferase® Reporter Assay system (Promega). Total luciferase counts for firefly and Renilla luciferase were measured 6 times at 10 sec intervals while they decayed. These values were converted to total counts per minute and averaged. Transfection experiments and luciferase assays were carried out at least 3 times for each cell line.

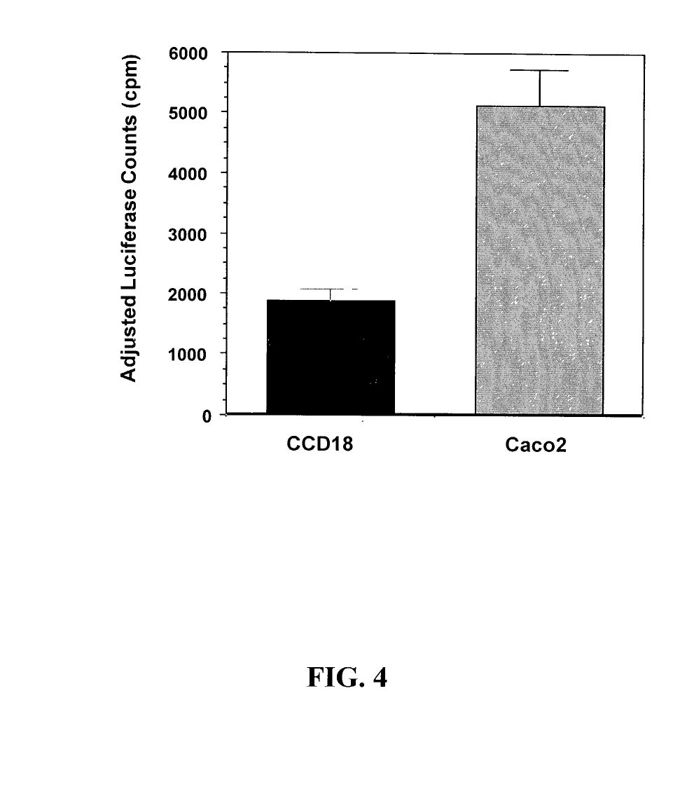

Referring now to FIG. 4 which depicts a bar diagram of the adjusted luciferase counts from extracts of CCD18 and Caco-2 cells corresponding to HMG-CoA reductase promoter activity in these two cell lines, respectively. The plasmid vector pRedLuc, which has HMG-CoA reductase promoter region fused with firefly luciferase reporter gene, was co-transfected into cells with a control vector, pRL-SV40 containing cDNA encoding Renilla luciferase. Firefly luciferase counts were recalculated by normalizing transfection efficiency with Renilla luciferase counts. It was found that the tumor cell line, Caco-2, generally had a higher transfection efficiency (3 to 10 times, measured by Renilla activity) than did the non-tumor cell line, CCD18, in each experiment. Total firefly luciferase counts in CCD18 were multiplied by a transfection efficiency factor and compared to total firefly luciferase counts in Caco-2 (FIG. 4). Firefly luciferase activity in Caco-2 cells was approximately 3 fold greater than that in CCD18, demonstrating that tumor cell HMG-CoA reductase promoter activity was elevated compared to normal cells. This result explains in part the enhanced HMG-CoA reductase mRNA levels found in tumor cells. The remainder could be related to greater HMG-CoA reductase mRNA stability in tumor cells.

### EXAMPLE 3

The effects of isoprenoids on reductase synthesis and mRNA levels were next studied Monoterpenes were found to regulate HMG-CoA reductase at the translational level. As discussed above, the end products of plant mevalonate metabolism, i.e., isoprenoids, suppress mammalian HMG-CoA reductase. To characterize the level at which plant-derived isoprenoids regulate reductase activity, lovastatin treated Syrian Hamster C100 cells were incubated with mevalonate or an isoprenoid (limonene, perillyl alcohol, or geraniol). These monoterpenes were selected because they are representative of isoprenoids found in a wide variety of plants, fruits and grains. C100 cells in which endogenous mevalonate biosynthesis is blocked with lovastatin have been a useful system to evaluate the effects of oxysterols and mevalonate-derived isoprenoids, either alone or in combination, on HMG-CoA reductase levels and rates of synthesis or degradation. Mevalonate decreased both reductase synthesis and mRNA levels by 65%. Under these conditions, decreased mRNA levels could be attributed to endogenously synthesized sterols. Both cyclic monoterpenes (limonene and perillyl alcohol) lowered reductase synthesis by 70 and 89%, respectively. However neither limonene nor perillyl alcohol had a significant impact on HMG-CoA reductase mRNAlevels. Geraniol, an acyclic monoterpene alcohol, suppressed reductase synthesis by 98% and lowered the mRNA level by 64%, respectively. The effects of the three isoprenoids on reductase degradation were also determined by measuring the half-life of HMG-CoA reductase. Neither limonene nor geraniol affected the half-life of reductase when added to lovastatin-treated SV28 cells. This was in contrast to mevalonate which decreased the HMG-CoA reductase half-life by approximately 60%. From these results, we concluded that the three plant-derived isoprenoids suppress reductase synthesis at a translational level.

In addition, geraniol attenuated reductase synthesis by decreasing HMG-CoA reductase mRNA levels. This dual action of geraniol may be of physiological significance as, analogous to farnesol, its diphosphate ester is an intermediate in the sterologenic pathway. Presently, it is unknown if geraniol mimics a side chain sterol and directly affects reductase 5′-SRE promoter elements. Alternatively, geraniol could inhibit a second enzyme of cholesterol biosynthesis such as squalene cyclase. These results with geraniol are significant because they illustrate that isoprenoids may have diverse effects on reductase translation, protein degradation, mRNA stability, or transcription depending on unique features of their structures.

TABLE 1
Effects of Isoprenoids on Reductase Synthesis and mRNA levels

| | Rate of | | |
|---|---|---|---|
| Treatment | Synthesis^{a} | mRNA Levels^{b} | Half-life (hr)^{c} |
| Lovastatin | 100 ± 15% | 100 ± 5% | 10.0 |
| Lovastatin + | 35 ± 5% | 34 ± 3% | 4.0 |
| Mevalonate (10 mM) | | | |
| Lovastatin + | 30 ± 6% | 115 ± 7% | 10.5 |
| limonene (5 mM) | | | |
| Lovastatin + | 11 ± 3% | 112 ± 5% | 16.0 |
| perillyl alcohol (0.7 mM) | | | |
| Lovastatin + | 1.4 ± 0.3% | 34 ± 6% | 9.6 |
| geraniol (0.4 mM) | | | |
| ^{a}Rates of reductase synthesis were based on the dpm of ³⁵S-labeled immunoprecipitable reductase from 1 × 10⁶ cells following a 1 hr labeling period. Values were corrected for incorporation of ³⁵S-methionine based on the amount of TCA precipitable ³⁵S-labeled | | | |
| # protein for each treatment condition. Rates of reductase synthesis in lovastatin-treated C100 cells were set at 100% and all other values are reported as a percentage (mean ± S.D.) of this value. | | | |
| ^{b}Reductase RNA values were corrected for RNA loading effects based on the amount of ribosomal protein (RP) S17 mRNA. Corrected reductase RNA values in lovastatin-treated cells were set at 100% and all other values are reported as a percentage of this value. | | | |
| ^{c}SV28 cells were labeled with ³⁵S-methionine for 1 hour in DFBS-MEM (without methionine) containing 25 μM lovastatin. The medium was then changed to DFBS-MEM containing methionine supplemented with lovastatin, mevalonate, limonene, | | | |
| # perillyl alcohol, or geraniol as indicated in the Table 1. Labeled reductase was measured by immunoprecipitation. | | | |

### EXAMPLE 4

It was found that eIF4E overexpression in Chinese hamster ovary (CHO) fibroblasts increases HMG-CoA reductase mRNA synthesis by attenuating translational suppression. It is known that eIF4E is the rate-limiting factor of the mRNA cap-binding complex or eIF-4F. Its overexpression leads to increased translational efficiency of many mRNAs coding for proteins involved in promotion of cell growth and proliferation. Such mRNAs share common features that include GC-rich 5′-UTL sequences with extensive secondary structure. As described above, HMG-CoA reductase mRNA contains these two common features. We have previously determined that HMG-CoA reductase translation is inefficient and regulated by mevalonate-derived nonsterols at the level of initiation. Therefore, we hypothesized that secondary structure plays a functional role in HMG-CoA reductase translational control.

To test this hypothesis, we developed a CHO cell line, rb4E, that overexpresses eIF4E by transfecting CHO cells with a retroviral vector, pMV7-4E, containing the full length coding cDNA sequence for mouse eIF4E. Control vectors included empty vector pMV7-neo and pMV7-4E(ALA), a vector containing a eIF4E cDNA in which serine 53 was replaced with alanine. Both eIF4E mRNA and protein levels were elevated five to ten-fold compared to nontransfected CHO cells (not shown). The rate of HMG-CoA reductase synthesis was then evaluated by immunoprecipitation and a rat HMG-CoA reductase-specific antibody, in cells expressing either eIF4E, eIF4E(ALA), or empty vector sequences.

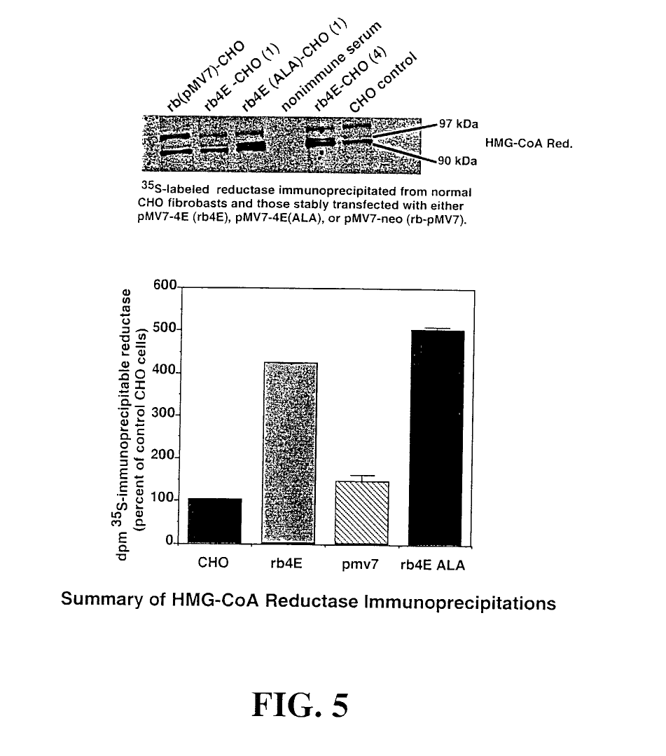

Referring now to FIG. 5 which depicts ³⁵S-labeled reductase immunoprecipitated from normal CHO fibrobasts and those stably transfected with either pMV7-4E (rb4E), pMV7-4E(ALA), or pMV7-neo (rb-pMV7). Immunoprecipitated HMG-CoA reductase was analyzed by SDS-PAGE and HMG-CoA reductase visualized by fluorography. In these autoradiographs (FIG. 5), HMG-CoA reductase appears as a doublet; the upper band represents the 97 kDa species associated with the endoplasmic reticulum and the smaller 90 kDa band is the species reported to be associated with peroxisomes. Both HMG-CoA reductase species were increased in rb4E-CHO(4) and rb4E(ALA) cells. The identity of the larger protein species migrating slower than the 97 kDa HMG-CoA reductase species is unknown, although its expression does not appear to be modulated by eIF4E expression. Phosphorimager analysis revealed that the 97 kDa species was increased by an average of 5-fold in both rb4E-CHO(4) and rb4E(ALA) cells compared to that in normal CHO cells. The 90 kDa species increased approximately 2 fold in rb4E and rb4E(ALA) cells. In addition, there was a 2-fold increase for HMG-CoA reductase in cells transfected with the empty vector sequence, pMV7-neo. There was no significant difference in general protein synthesis between any of these cell types, which was only increased by 15% in rb4E cells (data not shown). This was expected because eIF4E over-expression only affects a specific subset of mRNAs with complex 5′-UTL sequence structure. Although Ser53 was thought originally to be important in facilitating binding to the m⁷GTP cap of mRNA, the Ser53 to Ala mutant eIF4E also mediated an increase in HMG-CoA reductase equivalent to that of normal eIF4E. This site was recently ruled out as the major phosphorylation site(s), which has now been identified as Ser209. HMG-CoA reductase RNA levels were also measured in these cell lines by RPA (not shown). HMG-CoA reductase mRNA levels were decreased consistently by 2-fold in both rb4E and rb4E(ALA) compared to CHO cells. The decrease may relate to enhanced eIF4E-mediated mRNA translation, which is linked to enhanced mRNA degradation. Alternatively, this decrease may relate to greater sterol production in these transfected cells because of increased HMG-CoA reductase activity.

### EXAMPLE 5

The isoprenoid perillyl alcohol was found to regulate HMG-CoA reductase at the translational level by modulating 4E-BP1 phosphorylation. As discussed above, the P13-kinase pathway has a critical role in regulating translation of mRNAs with GC-rich 5′-UTLs. Translation of these mRNAs is highly dependent on eIF4E levels. Regulation of eIF4E available for formation of the m⁷GTP cap binding complex, eIF4F, occurs in part through regulated binding to 4E-BP1. In response to mitogens, 4E-BP1 is phosphorylated and releases eIF4E. Increased cellular eIF4E levels permit translation of specific mRNAs with highly structured 5′-UTLs. Human 4E-BP1 is phosphorylated at six sites in a step wise fashion (T37, T46, S65, T70, S83, and S112). Changes in the relative amounts of hyperphosphorylated and hypophosphorylated 4E-BP1 isoforms can be used to monitor changes in P13 kinase signal transduction activity. Moreover, overexpression of prenylated Rab family members has been correlated with metastatic potential of lung cancer.

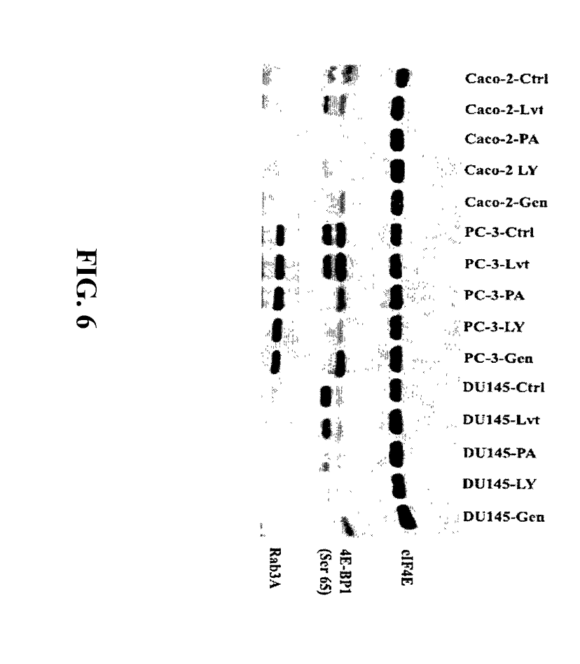

We treated PC-3, DU145, and Caco-2 (colon adenocarcinoma) cells with lovastatin (LVT), perillyl alcohol (PA), the P13 kinase inhibitor, LY294002 (LY), or genistein (Gen). PC-3, DU145, and Caco-2 cells were plated in RPMI plus 10% FBS and cells allowed to grow for 24 hr. Medium was supplemented with lovastatin (1 μM for 16 hr), perillyl alcohol (400 μM for 16 hr), genistein (40 μM for 4 hr), or LY 294002 (10 μM for 4 hr). Cells were lysed in RIPA buffer and protein extracts quantitated. Aliquots (100 μg) were electrophoresed and transferred to PVDF membranes. Cells were lysed and proteins separated by 10% SDS PAGE and transferred to PVDF membranes. The eIF4E (FIG. 6, top panel) and 4E-BP1 (FIG. 6, middle panel) were detected using antibodies specific for eIF4E and phospho-4E-BP1 (Ser65). Specific proteins were detected by ECL using a Kodak Image Station.

Referring now to FIG. 6 which depicts a Western blot analysis of eIF4E, 4E-BP1 (Ser65) and Rab3A from various cells. The eIF4E showed no change with any of the treatments. The same blot was reprobed with phospho-4E-BP1 (Ser65). Perillyl alcohol reduced Ser65 phosphorylation in a manner analogous to LY294002 in both prostate cell lines indicating that perillyl alcohol acts through the (P13K-Akt-mTOR-eIF4E/4E-BP1) signal transduction pathway. Genistein did not appear to affect Ser65 phosphorylation of the upper band but reduced phosphorylation of the lower of the two bands. These results indicate that the receptor (EGFR-tyrosine kinase-Ras/Raf-MAPK (Erk1/2) signal transduction pathway may have an impact on 4E-BP1 phosphorylation. Treatment with the mTOR inhibitor rapamycin also suppressed Ser65 phosphorylation in both cell types (data not shown). These experiments indicate that perillyl alcohol decreases 4E-BP1 phosphorylation, an effect that would reduce the amount of eIF4E available for formation of the cap binding complex, eIF4F. This result is significant because reduced phosphorylation of 4E-BP1 at Ser65 is associated with reduced cap dependent translation and enhanced apoptosis. Therefore, growth suppression by perillyl alcohol could be attributed in part to this effect on the (P13K-Akt-mTOR-eIF4E/4E-BP1) signal transduction pathway. Rab3A levels were also compared in the highly metastatic PC-3 cells, DU145 and in Caco-2, which is considered to have low metastatic potential. The blot shown in FIG. 6 was reprobed with a Rab3A specific antibody (lower panel). Rab3A protein could not be detected in Caco-2 but was present in high levels in PC-3 cells. Levels of Rab3A were substantially lower in DU145 cells. Rab3A was undetectable in normal PrEC cells (data not shown). Perillyl alcohol was found to further reduce the levels of Rab3A protein in DU145 cells. These results provide strong evidence for a link between Rab3A prenylation, increased protein secretion and enhanced invasive capacity.

### EXAMPLE 6

Elevated eIF4E is associated with many tumor cell types and is thought to contribute to rapid and uncontrolled cell proliferation. Accelerated proliferation is thought to be due in part to enhanced and aberrant translation of mRNAs coding for growth and cell cycle regulatory proteins. Therefore, standard Western blot procedures were used to compare eIF4E levels in prostate cancer cells versus normal PrEC cells.

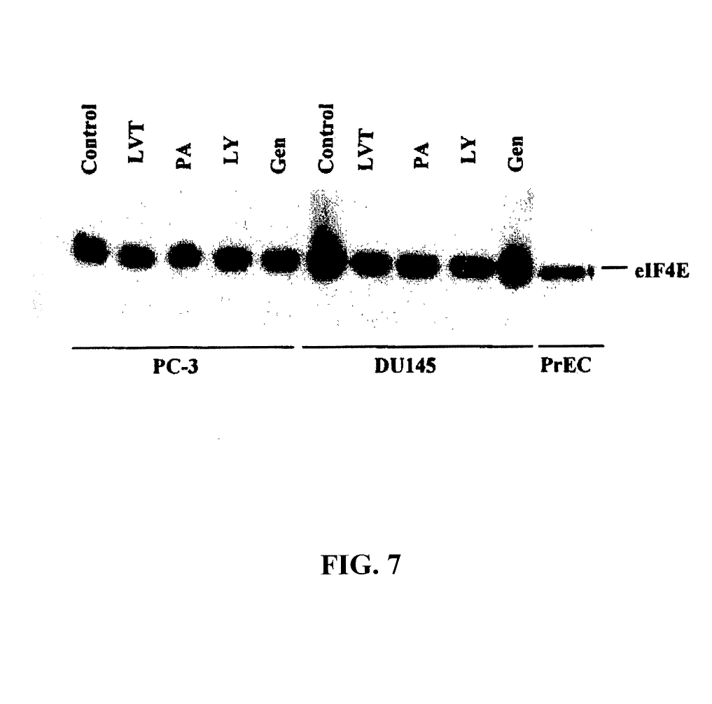

Referring now to FIG. 7 which depicts a standard Western blot analysis of the eIF4E associated with PC-3, DU145 and PrEC cell lines. Greatly elevated eIF4E levels were found in human prostate cancer cells relative to normal PrEC. Sample preparation conditions were those as described above in Example 6.

### EXAMPLE 7

Genistein was found to suppress HMG-CoA reductase mRNA in cells. Quantitative RT-PCR (described above) was used to measure changes in DU145 HMG-CoA reductase mRNA following treatment with genistein (40 μM) or the MEK1 inhibitor, PD98059 (50 μM). Following treatment for 16 hr, cells were lysed, RNA prepared, and HMG-CoA reductase mRNA copy number determined using an internal pAW109 RNA mimic. Untreated cells had approximately 5×10⁶ copies/μg of RNA. Treatment with genistein or PD98059 had similar effects on HMG-CoA reductase mRNA, reducing its copy number to approximately 2.5 to 3.0×10⁶ copies/μg RNA. This provides evidence that genistein can control mevalonate synthesis by modulating HMG-CoA reductase mRNA levels in human prostate tumor cells. Combined with data showing that pure isoprenoids affect HMG-CoA reductase translation, these observations form part of the basis of the proposed mechanism of synergistic interaction.

### EXAMPLE 8

Isoprenoids were found to elicit differential effects on MMP activation processes in tumor cell lines. Since the effects of isoprenoids with or without genistein on prostate cancer metastasis had not been explored prior to the present invention, the effects of genistein, perillyl alcohol, and γ-tocotrienol were studied on MMP-9 and MMP-2 activity. MMP activity as described in the background has been linked in part to the Ras/ERK pathway and may also be involved in a second unidentified pathway that is ERK-independent. Therefore, we employed gelatin zymography to study effects of isoprenoids on MMP-9 and MMP-2 activation and secretion.

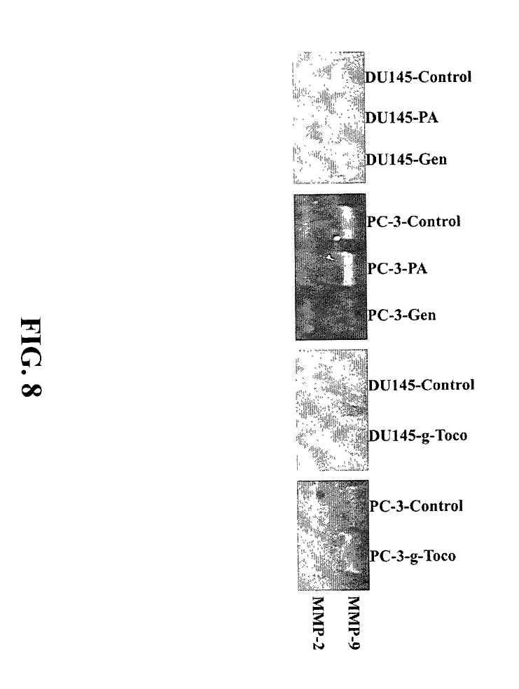

Referring now to FIG. 8. which depicts a gelatin zymogram showing the effects of perillyl alcohol (PA), genistein (Gen), or γ-tocotrienol (g-Toco) on MMP activity. The results of FIG. 8 are quantified in Table 2. Cells were grown in 100 mm culture plates to approximately 80% confluency. Prior to experimental treatments, cells were washed extensively in serum-free medium and then incubated for 24 hr in serum-free media with Mito plus® serum supplement (Becton-Dickinson) in the absence or presence of isoprenoids. Conditioned media were then collected from PC-3 and DU145 cells that were incubated in the absence or presence of genistein (20 μM), perillyl alcohol (400 μM), or γ-tocotrienol (20 μM) for 16 hr and clarified by centrifugation. MMP activity in conditioned media was analyzed by mixing aliquots with SDS sample loading buffer without β-mercaptoethanol (non-reducing). Samples were electrophoresed on a 10% polyacrylamide (29:1) separating gel containing 0.1% (w/v) gelatin. Gels were incubated in developing buffer at 37° C. overnight. Enzyme activity was observed after staining with Coomassie Blue as clear bands on a blue background of undigested gelatin. Band density was then normalized to cell number prior to- and post-treatment (See FIG. 8).

It was found that perillyl alcohol nearly extinguished all MMP-9 activity from the DU145 cells but exhibited no similar effect in PC-3 cells. Conversely, genistein was found to suppress MMP-9 activity by nearly ninety percent in PC-3 cells but exhibited no similar effect in DU145. The γ-tocotrienol treatments of both DU145 and PC3 exhibited modest monotonic decreases in both MMP-9 and MMP-2. These results suggest that MMP activity in prostate carcinomas is regulated by different signaling pathways in different tumor cell lines and thus require empirical measurements to evaluate exactly how isoprenoids differentially effect this activity process. These findings graphically emphasize the utility of the multi-isoprenoid approach for treating cancer by inhibiting multiple biomechanical processes associated with carcinoma in a living organism.

TABLE 2
Effects of Isoprenoids on Human Prostate Cell MMP production.

| Cell Line | Condition | MMP-9 Count | MMP-2 Count |
|---|---|---|---|
| DU145 | CONTROL | 30.71 | 15.05 |
| DU145 | Perillyl Alcohol | 0.09 | 10.63 |
| DU145 | Genistein | 47.64 | 23.54 |
| DU145 | CONTROL | 23.5 | 5.5 |
| DU145 | γ-tocotrienol | 16.5 | −2.2 |
| PC-3 | CONTROL | 46.95 | 11.85 |
| PC-3 | Perillyl Alcohol | 72.65 | 6.73 |
| PC-3 | Genistein | 5.25 | 4.25 |
| PC-3 | CONTROL | 18.14 | 18.14 |
| PC-3 | γ-tocotrienol | 27.4 | 15.3 |

### EXAMPLE 9

The Membrane Invasion Culture System (MICS) represents a direct measurement of intracellular reorganization and invasive capability of cancer cell lines. The MICS protocol was used to quantitate the effects of isoprenoid treatment on the invasive potential of PC-3 prostate cancer cells. Polycarbonate membranes (10 μm pores) were coated with a basement membrane matrix (MATRIGEL™, BD Biosciences) and placed inside MICS chambers. Both upper and lower chambers were filled with serum-free RPMI 1640 containing Mito plus™. Cells were seeded onto the upper wells at 1×10⁵ cells/well. After 24 hr, cells that had invaded through the membrane were collected and counted. Invasion rates were calculated as the percent cells invading the matrix coated membrane compared to total number of cells seeded (Table 3).

Referring now to Table 3, the effects of isoprenoids on inhibiting invasion are summarized. Each isoprenoid alone (i.e., perillyl alcohol, genistein, and y-tocotrienol) inhibited the invasive ability of PC-3 cells, with genistein exhibiting the greatest effect. The combination of perillyl alcohol plus genistein or γ-tocotrienol plus genistein did not provide any greater effect against invasion.

TABLE 3
Effects of Isoprenoids on Inhibiting Invasion

| Cell Line | Treatment Condition* | percent invaded |
|---|---|---|
| PC-3 | CONTROL | 12.5% |
| PC-3 | Perillyl Alcohol | 5% |
| PC-3 | Genistein | 0% |
| PC-3 | Perillyl Alcohol & Genistein | 3% |
| PC-3 | γ-tocotrienol | 7% |
| PC-3 | γ-tocotrienol & Genistein | 7% |

*Perillyl Alcohol at 400 μM, Genistein at 20 μM and γ-tocotrienol at 20 μM.

### EXAMPLE 10

An in vitro collagen gel contraction assay was used to evaluate the effects of isoprenoids on biomechanical tumor cell matrix remodeling from various prostate cancer cell lines. Collagen gel suspensions for each tumor cell line were prepared by mixing a 250 μl suspension of 3×10⁶ cells/ml with 250 μl of undiluted rat tail collagen type I (BD Biosciences). The resultant collagen gel suspensions were dripped onto 35 mm petri dishes containing 3 ml RPMI 1640 in 10% FBS to prevent adhesion of collagen to the dish. Collagen gel suspensions were allowed to polymerize for one hr. Isoprenoid treatments were added 24 hr after exchanging culture medium and were removed 24-48 hr post treatment. Cultures were observed for 2-3 wk to determine the degree of contraction, wherein the degree of contraction was defined as the relative change in gel diameter over time.

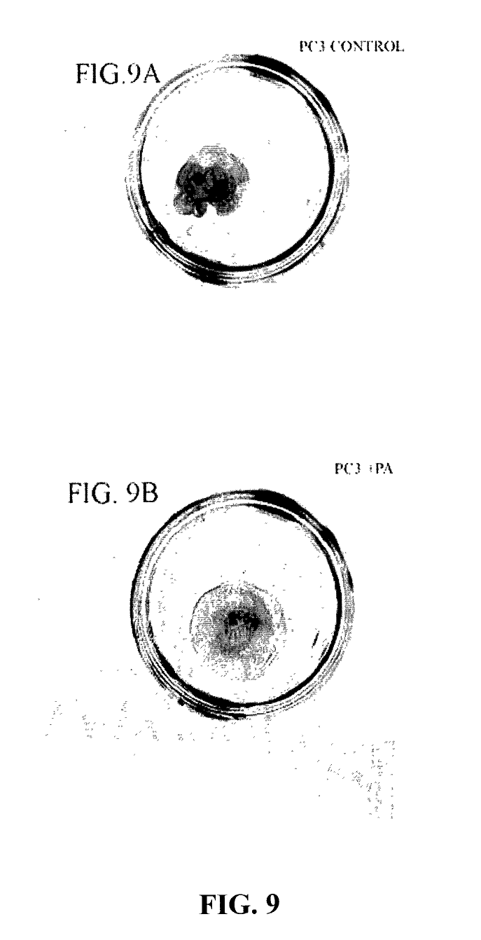

Photographic results of the in vitro collagen gel contraction assays are presented in FIG. 9 which visually depicts how perillyl alcohol differentially inhibited the extracellular matrix remodeling tendencies of PC3 cells by maintaining the size and shape of the collagen gel balls containing this carcinoma. FIG. 9A depicts considerable matrix remodeling of the control gel ball containing PC3 cells, whereas FIG. 9B shows PA (i.e., perillyl alcohol) retards matrix remodeling of the gel ball containing PC3 cells. Table 4 summaries the effects on collagen gel ball diameters for both PC3 and DU145 as a result from being exposed to various isoprenoids during the in vitro collagen gel contraction assay.

TABLE 4
Effects of isoprenoids on matrix remodeling

| Cell Line | Condition* | Diameter (cm) |
|---|---|---|
| DU145 | CONTROL | 3.0 |
| DU145 | Perillyl Alcohol | 3.75 |
| DU145 | Genistein | 3.2 |
| PC-3 | CONTROL | 2.0 |
| PC-3 | Perillyl Alcohol | 3.2 |
| PC-3 | Genistein | 3.5 |

*Perillyl Alcohol at 400 μM, and Genistein at 20 μM

As to the manner of usage and operation of the present invention, the same should be apparent from the above description. Accordingly, no further discussion relating to the manner of usage and operation will be provided.

While preferred embodiments of the method and associated kit for treating cancer have been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Throughout this specification, unless the context requires otherwise, the word “comprise” or variations such as “comprises” or “comprising” or the term “includes” or variations, thereof, or the term “having” or variations, thereof will be understood to imply the inclusion of a stated element or integer or group of elements or integers but not the exclusion of any other element or integer or group of elements or integers. In this regard, in construing the claim scope, an embodiment where one or more features is added to any of the claims is to be regarded as within the scope of the invention given that the essential features of the invention as claimed are included in such an embodiment.

Those skilled in the art will appreciate that the invention described herein is susceptible to variations and modifications other than those specifically described. It is to be understood that the invention includes all such variations and modifications which fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1. A method of treating cancer by inhibiting at least one biomechanical process associated with a carcinoma in a living organism, said method comprising the step of exposing the carcinoma to an effective amount of an isoprenoid, wherein said effective amount of isoprenoid inhibits the biomechanical process associated with the carcinoma.

2. The method of claim 1 wherein said effective amount of isoprenoid inhibits the biomechanical process comprising overproduction of HMG-CoA reductase mRNA.

3. The method of claim 1 wherein said effective amount of isoprenoid inhibits the biomechanical process comprising enhanced translation of HMG-CoA reductase from its mRNA template.

4. The method of claim 1 wherein said effective amount of isoprenoid inhibits the biomechanical process comprising elevated 4E-BP1 phosphorylation.

5. The method of claim 1 wherein said effective amount of isoprenoid inhibits the biomechanical process comprising overexpression of eIF4E.

6. The method of claim 1 wherein said effective amount of isoprenoid inhibits the biomechanical process comprising increased levels and activity of matrix metalloproteinases (MMPs).

7. The method of claim 6 wherein said matrix metalloproteinases comprise MMP-2.

8. The method of claim 6 wherein said matrix metalloproteinases comprise MMP-9.

9. The method of claim 1 wherein said effective amount of isoprenoid inhibits the biomechanical process comprising elevated Rab3A levels.

10. The method of claim 1 wherein said effective amount of isoprenoid inhibits the biomechanical process comprising extracellular matrix remodeling.

11. The method of claim 1 wherein said effective amount of isoprenoid inhibits the biomechanical process comprising secondary tissue invasion.

12. The method of claim 1 wherein the isoprenoid is in a delivery device selected from the group consisting of a tablet, a capsule, a solution, a suspension, an emulsion, a foodstuff, a pharmaceutical preparation, a nutritional supplement, and a dietary additive.

13. The method of claim 1 wherein said exposing step is selected from the group consisting of contacting directly the isoprenoid onto the carcinoma in the living organism, administering the isoprenoid intravenously in the living organism, injecting the isoprenoid intraperitoneally in the living organism, applying the isoprenoid subcutaneously in the living organism, inserting the isoprenoid intramuscularly in the living organism, employing the isoprenoid intrathecally in the living organism, swallowing the isoprenoid orally by the living organism, introducing the isoprenoid rectally into the living organism, rubbing the isoprenoid topically onto the living organism, and inhaling the isoprenoid by the living organism.

14. The method of claim 1 wherein the cancer is selected from the group consisting of cancers of the central nervous system, gastrointestinal tract, epidermal system, head and neck system, genitourinary tract, lymphatic system, cardiovascular system, hepatic system and respiratory system.

15. The method of claim 1 wherein the living organism is selected from the group consisting of a cell line, a mouse, a rat, a cat, a dog, a pig, a goat, a sheep, a cow, a horse, a monkey and a human.

16. The method of claim 1 wherein said exposing step is performed daily.

17. The method of claim 1 wherein the anti-neoplastic effective amount of the isoprenoid comprises about one nanogram to about fifty grams.

18. A method of treating cancer by inhibiting at least one biomechanical process associated with a carcinoma in a living organism, said method comprising the step of exposing the carcinoma to an effective amount of a composition containing a first isoprenoid and a second isoprenoid, wherein said effective amount of the composition containing the first isoprenoid and the second isoprenoid inhibits the biomechanical process associated with the carcinoma.

19. The method of claim 18 wherein the first isoprenoid and the second isoprenoid in the composition of said exposing step act additively together in the effective amount of the composition.

20. The method of claim 18 wherein the first isoprenoid and the second isoprenoid in the composition of said exposing step act synergistically together in the effective amount of the composition.

21. The method of claim 18 wherein the first isoprenoid and the second isoprenoid in the composition of said exposing step act antagonistically together in the effective amount of the composition.

22. A kit for using a method of treating cancer by inhibiting at least one biomechanical process associated with a carcinoma in a living organism, said kit comprising an effective amount of a composition containing a first isoprenoid and a diluent, wherein said effective amount of the composition inhibits the biomechanical process associated with the carcinoma; and a delivery device.

23. The kit of claim 22 wherein said delivery device is selected from the group consisting of a tablet, a capsule, a solution, a suspension, an emulsion, a foodstuff, a pharmaceutical preparation, a nutritional supplement, and a dietary additive.

24. The kit of claim 22 wherein said composition further comprising a second isoprenoid.
